(19) United States
(12) Patent Application Publication
(10) Pub. No.: US 2011/0236467 A1
(43) Pub. Date: Sep. 29, 2011

(54) METHOD FOR ERECTILE FUNCTION POTENTIATION BY PHARMACEUTICAL COMPOSITIONS COMPRISING TOXIN TX2-6 OF THE SPIDER PHONEUTRIA NIGRIVENTER

(76) Inventors: Maria Elena de Lima Perez Garcia, Belo Horizonte MG (BR); Rubén Dario Sinisterra Millán, Belo Horizonte MG (BR); Kênia Pedrosa Nunes, Belo Horizonte MG (BR); Rômulo Leite, Divinopolis MG (BR); Luciana Franco Lanza, Belo Horizonte MG (BR); Marcelo Ribeiro Vasconcelos Diniz, Belo Horizonte MG (BR); Marta do Nascimento Cordeiro, Belo Horizonte MG (BR); Michael Richardson, Belo Horizonte MG (BR); Ana do Carmo Valentim, Belo Horizonte MG (BR); Adriano Monteiro de Castro Pimenta, Belo Horizonte MG (BR)

(21) Appl. No.: 12/865,818

(22) PCT Filed: Jan. 30, 2009
(86) PCT No.: PCT/BR2009/000040

§ 371 Date: May 6, 2011

(30) Foreign Application Priority Data
Jan. 31, 2008 (BR) PI0800596-6

(51) Int. Cl. A61K 9/127; C07K 14/435; C07H 21/04; A61K 38/17; A61P 15/00

(52) U.S. Cl. 424/450; 530/324; 536/23.5; 514/21.3

(57) ABSTRACT

The present invention concerns a method for male erectile function potentiating by means of toxin Tx2-6 from the spider Phoneufria nigriventer.

The present invention concerns a method for the potentiation of male penile erectile function by using pharmaceutical compounds from the toxin Tx2-6 of Phoneutria nigriventer spider. The use of pharmaceutical compounds from the Tx2-6 toxin is claimed here as a method for recovering of erectile function of patients suffering from erectile dysfunction, as for example hypertensive individuals.

Penile erection is a complex hemodynamic function that occurs under controlled regulation. It is initiated through the activation of parassympatic pelvic nerves leading to arterial dilation followed by relaxation of the corpus cavernosum (ANDERSSON K. E., WAGNER G. Physiology of penile erection. Physiol Rev 75, 191-236, 1995).

There is a scientific consensus that nitric oxide (NO) is a prerequisite for generating and maintaining increased intracavernosal pressure and penile erection. The NO synthesis is accomplished by the nitric oxide synthase (NOS) enzyme. The constitutive forms of this enzyme—neuronal NOS (nNOS) and endothelial NOS (eNOS)—are involved in the induction of penile erection (IGNARO, L. J., BUSH, P. A., BUGA, G. M., WOOD, K. S., FUKUTO, J. M., RAIFER, J. Nitric oxide and cyclic GMP formation upon electrical field stimulation cause relaxation of corpus cavernosum smooth muscle. Biochem Biophys Res Commun 170, 843-850, 1990).

NO is released by the nitrergic nerves in the trabecular and neuronal tissues of penile arteries (KIM, N., AZADZOI, K. M., GOLDSTEIN, I., SAENZ DE TEJADA, I. A nitric oxide-like factor mediates nonadrenergic-noncholinergic neurogenic relaxation of penile corpus cavernosum smooth muscle. J Clin Invest 88, 112-118, 1991), by exerting their relaxing action in the corpus cavernosum arteries through the activation of the guanylate cyclase, which increases the intracellular concentration of cGMP (cyclic guanosine monophosphate) (MIZUSAWA, H., HEDLUND, P., BRIONI, J. D., SULLIVAN, J. P., ANDERSON, K. E. Nitric oxide independent activator of guanylate cyclase by YC-1 causes erectile responses in the rat. J Urol, 167, 2276-2281, 2002).

The cynase I protein, which is dependent on cGMP (cGK1 or PKG), may subsequently alter the intracellular calcium channels and through the opening of calcium-dependent K+ channels lead to a hyperpolarization of smooth muscle cells (CHRIST, G. J., WANG, H. Z., VENKATESWARLU, K., ZHAO, W., DAY N. S. Ions channels and gap junctions: their role in erectile physiology, dysfunction, and future therapy. Mol. Ural., 3, 61-73, 1999). PKG may phosphorylate other proteins affecting calcium channels or change the phosphorylation status of the myosin light chain (MLC), resulting in cavernosal muscle relaxation mediated by NO (MILLS, T. M., CHITALEY, K., WINGARD, C. J., LEWIS, R. W., WEBB, R. C. Effect of Rho-kinase inhibition on vasoconstriction in the penile circulation. J Appl Physiol, 91, 1269-1273, 2001).

Erectile dysfunction (ED) occurs due to unbalanced contracting and relaxing factors within the corpus cavernosum, especially through a hindrance of the NO system, hypertension being an ED risk factor (ANDERSSON K. E. Pharmacology of penile erection. Pharmacol Ver 53, 417-450, 2001). Stimulation of the cavernosal nerves in anesthetized animals elicits penile erection due to an increased intracavernosal pressure mediated NO (BURNETT, A. L., CHANG, A. G., CRONE, J. K., HUANG, P. L., SEZEN, S. F. Noncholinergic penile erection in mice lacking the gene for endothelial nitric oxide synthase. J. Androl., 23, 92-97, 2002).

The venom of the spider Phoneutria nigriventer is a rich source of bioactive peptides (CORDEIRO, M. N., RICHARDSON, M., GILROY, J., FIGUEIREDO, S. G. D., BEIRÃO, P. S. L., DINIZ, C. R. Properties of the venom from the South American armed spider Phoneutria nigriventer (Keyserling, 1891). J. Toxicol—Toxin Rev., 14, 309-326, 1995; RICHARDSON, M., PIMENTA, A. M. C., BEMQUERER, M. P., SANTORO, M. M., BEIRAO, P. S. L., De LIMA, M. E., FIGUEIREDO, S. G., BLOCH, C. Jr., VASCONCELOS, E., CAMPOS, F. A. P., GOMES, P. C., CORDEIRO, M. N. Comparison of the partial proteomes of the venoms of Brazilian spiders of the genus Phoneutria. Comp. Biochem. Physiol. C, Comp. Pharmacol. Toxicl 142, 173-187, 2006), and this spider accounts for serious human accidents characterized by different symptoms (BUCARETCHI, F., DEUS REINALDO, C. R., HYSLOP, S., MADUREIRA, P. R., De CAPITANI, E. M., VIEIRA, R. J. A clinico-epidemiological study of bites by spiders of the genus Phoneutria. Rev. Inst. Med. Trop, Sao Paulo 42, 17-21, 2000), including penile erection (YONAMINE, C. M., TRONCONE, L. R. P., CAMILLO, M. A. P. Blockade of neuronal nitric oxide synthase abolishes the toxic effects of Tx2-5, a lethal Phoneutria nigriventer spider toxin. Toxicon 44 169-172, 2004). The P. nigriventer venom contains several neurotoxins that exert various biological effects. Ionic channels, such as sodium, are usually the targets involved in such effects (ARAÚJO, D. A, CORDEIRO, M. N., DINIZ, C. R., BEIRÃO, P. S. L. Effects of a toxic fraction, PhTx2, from the spider Phoneutria nigriventer on the sodium current. Naunyn-Schmiedeberg's Arch Pharmacol, 347, 205-208, 1993; MARTIN-MOUTOT, N., MANSUELLE, P., ALCARAZ, G., DOS SANTOS, R. G., CORDEIRO, M. N., DE LIMA, M. E., SEAGAR, M. & VAN RENTERGHEM, C. Phoneutria nigriventer toxin 1: a novel, state-dependent inhibitor of neuronal sodium channels that interacts with micro conotoxin binding sites. Mol Pharmacol, 69, 1931-7, 2006), calcium (LEÃO, R. M., CRUZ, J. S., DINIZ, C. R., CORDEIRO, M. N., BEIRÃO, P. S. Inhibition of neuronal high-voltage activated calcium channels by the Phoneutria nigriventer Tx3-3 peptide toxin. Neuropharmacology, 39, 1756-1767, 2000; DOS SANTOS, R. G., VAN RENTERGHEM, C., MARTIN-MOUTOT, N., MANSUELLE, P., CORDEIRO, M. N., DINIZ, C. R., MORI, Y., DE LIMA, M. E. & SEAGAR, M. Phoneutria nigriventer omega-phonetoxin IIA blocks the Cav2 family of calcium channels and interacts with omega-conotoxin-binding sites. J Blol Chem, 277, 13856-62, 2002) and potassium (KUSHMERICK C, KALAPOTHAKIS E, BEIRAO P S, PENAFORTE C L, PRADO V F, CRUZ J S, DINIZ C R, CORDERO M N, GOMEZ M V, ROMANO-SILVA M A, PRADO M A. Phoneutria nigriventer toxin Tx3-1 blocks A-type currents controlling Ca²⁺oscillation frequency in GH3 cells, J. Neurochem, 72(4): 1472-81, 1999).

Toxin Tx2-6, a toxin of the spider P. nigriventer, is a polypeptide containing 48 amino acid residues, 10 out of them being cysteines (CORDEIRO, M. N., DINIZ, C. R., VALENTIN, A. C., VON EICKSTEDT, V. R., GILROY, J., RICHARDSON, M. The purification and aminoacid sequences of four Tx2 neurotoxins from the venom of the Brazilian “armed” spider Phoneutria nigriventer. FEBS Lett, 310(2):153-156, 1992). Reducing the swift inactivation of the voltage-dependent sodium channels is the major action of this toxin (MATAVEL, A., CRUZ, J. S., PENAFORTE, C. L., ARAUJO, D. A. M., KALAPOTHAKIS, E., PRADO, V. F., DINIZ, C. R., CORDEIRO, M. N., BEIRAO, P. S. L. Electrophysiological characterization and molecular identification of the Phoneutria nigriventer peptide toxin PnTx2-6. FEBS Letters, 532, 219-223, 2002), which is mediated—in this kind of toxin—by its action in site 3 of the sodium channel (Cestele, S. & Catterall, W. A. Molecular mechanisms of neurotoxin action on voltage-gated sodium channels. Biochimie, 82, 883-92, 2000).

The P. nigriventer crude venom induces cavernosal relaxing, which may be prevented by applying L-NAME (ANTUNES, E., MARANGONI, R. A., GIGLIO, J. R., BRAIN, S. D., de NUCCl, G. Activation of tissue kallikrein-kininogen-kinin system in rabbit skin by a fraction isolated from Phoneutria nigriventer (armed spider) venom. Toxicon, 31, 1385-1391, 1993).

U.S. Pat. No. 6,365,590 describes vasoactive compounds for treating erectile dysfunction and impotence. These compounds are products of the reaction of a negatively charged component able to induce erection (such as alprostadil) and a positively charged component (such as prazosin) or a local anaesthetic agent (such as lidocaine). These components are combined as acids and bases to form an organic salt or a compound with an ionic ligand.

U.S. Pat. No. 7,105,571 describes methods and compounds for treating erectile dysfunction. This method consists of inserting, within the patient's navicular pit, of an effective semi-solid compound containing vasoactive prostaglandin able to induce erection. This compound includes a vasoactive prostaglandin, an agent that facilitates the compound absorption, a polysaccharide, a lipophilic compound and a buffer system.

U.S. Pat. No. 6,291,471 describes the use of apomorfine for treating erectile dysfunction, particularly in those cases related to vascular alterations. A therapeutic method consisting of delivering a therapeutical amount of apomorfine or a prodrug of this pharmaceutical is also described.

U.S. Pat. No. 5,942,545 describes a compound and a method for treating penile erectile dysfunction. This invention makes use of a compound containing prostaglandin E1 through topic transdermal delivery on the penis. It is said to be nonirritating and efficacious in the treatment of penile erectile dysfunction. An effective amount of agents that promote absorption, such as dioxalone, dioxane or Carrier compounds based on ethanolic solution may also be used. Fentolamin or prazosin may be used combined with prostaglandin E1.

U.S. Pat. No. 6,586,391 describes a method for reducing erectile dysfunction by delivering an endothelin antagonist so as to promote vasodilatation via NO production.

U.S. Pat. No. 7,223,406 describes methods and compounds for preventing and treating erectile dysfunction. This invention is based on delivering an effective amount of one or more factors belonging to a group of substances, among them the vascular endothelial growth factor, brain-derived neurotrophic factor, fibroblast growth factor, neurotrophin-3, neurotrophin-4 or angiopoietin-1. The factor used may be an integral protein or a nucleic acid codifying the factor or a functional fragment of the protein. Combinations, kits and combinatory methods are also described.

U.S. Pat. No. 7,022,728 describes new derivatives of benzimidazol, which are useful for treating male or female sexual dysfunctions, Alzheimers disease, Parkinsons disease, schizophrenia, anxiety, humor and behavior disorders.

A pharmaceutical may be chemically modified so as to have its properties changed, such as its biodistribution, pharmacokinetics and solubility. Several methods have been used to increase solubility and stability of drugs, among them the use of organic solvents, emulsions, liposomes, pH adjustment, chemical alterations and complex formation of pharmaceuticals with an appropriate encapsulating agent, such as cyclodextrines. Cyclodextrines belong to the cyclic oligosaccharide family that includes six, seven or eight glucopyranose units. Due to steric interactions, cyclodextrines (CDs) produce a cyclic structure in the form of a truncated cone with an apolar internal cavity. These are chemically stable compounds that may be regioselectively modified.

Cyclodextrines (hosts) form complexes together with several hydrophobic molecules (guests), including these very molecules entirely or partially into CD's cavity. CDs have been used for solubilization and encapsulation of drugs, perfumes and aromatizers as described in Szejtli, J., Chemical Reviews, (1998), 98, 1743-1753; Szejtli, J., I. Mater. Chem., (1997), 7, 575-587. According to detailed studies of their toxicity, mutagenicity, teratogenicity and carcinogenicity, described in Rajewski, R. A., Stella, V., J. Pharmaceutical Sciences, (1996), 85, 1142-1169, cyclodextrines show low toxicity, especially that of hydroxypropyl-p-cyclodextrine, as reported in Szejtli, J. Cyclodextrins: Properties and applications. Drug Investig., (1990) 2(suppl. 4):11-21. Except for some highly concentrated derivatives, which elicit damage in erythrocytes, these products do not usually cause risks to health.

CDs are moderately soluble in water, methanol and ethanol and promptly soluble in aprotic polar solvents, such as dimethyl suffoxide, dimethylformamide, N,N-dimethylacetamide and piridine.

Numerous works can be found in the literature on the effects of increased solubility in water of less soluble guests, by using cyclodextrines via inclusion compounds as well as a discussion on the stability of inclusion complexes whose physicochemical features were well described in Szejtli, J., Chemical Reviews, (1998), 98, 1743-1753. Szejtli, J., J. Mater. Chem., (1997),7, 575-587. Cyclodextrines may be used for obtaining pharmaceutical formulations with peptides and/or proteins with a view to improving their stability and bioavailability.

In this way, the present invention has used the strategy of supramolecular compound formation between Tx2-6 and cyclodextrines as an example of a pharmaceutical compound used in tests for erectile dysfunction of normotensive and hypertensive rats.

In addition to cyclodextrines, biodegradable polymers, mucoadhesive polymers and gels are also used as devices for a Tx2-6 toxin controlled release. In such systems, the toxin is imbedded in a polymeric matrix based on a microspheric drug encapsulation, which releases the drug within the organism in small and controlled daily doses during several days, months or even years. As for gels, formulations can be used topically.

Several polymers have been tested in systems of controlled release, due to their physical properties, namely: poly(urethans) for their elasticity, poly(siloxanes) or silicones for their good isolation, poly(methyl-metacrilates) for their physical strength, poly(alcohol vinyls) for their hydrophobicity and resistance, poly(ethylenes) for their hardness and impermeability (Gilding, D. K. Biodegradable polymers. Biocompat. Clin. Implat. Mater. (1981) 2:209-232).

However, for human usage, the material must be chemically inert and free from impurities. Some materials used in release systems were: poly(2-hydroxy-ethylmetacrilate), polyacrylamide, polymers in a lactic acid base (PLA), glycolic acid base (PGA), and respective co-polymers, (pLGA) and poly(anhydrides), such as polymers in sebacic acid base (PSA) and co-polymers with more hydrophobic polymers.

The present invention formulation is characterized by the use of a mixture of pharmaceutically acceptable excipients combined with Tx2-6 and its pharmaceutically acceptable salts included in cyclodextrines and at least another pharmaceutically active compound included or not in cyclodextrines, liposomes and microencapsulates in biodegradable polymers, such as: PLA, PLGA and/or their mixtures. Formulations can be prepared with an excipient or a mixture of excipients. Examples of excipients are as follows: water, saline solution, phosphate buffered solutions, Ringer solution, dextrose solution, Hank solution, biocompatible saline solutions containing or not glycol polyethylene. Nonaqueous vehicles, such as fixed oils, sesame oil, ethyl-oleate or triglyceride, can also be used. Other useful formulations include agents capable of increasing viscosity, such as sodium carboxymethylcellulose, sorbitol or dextran for obtaining gels or mucoadhesive formulations, which facilitate their delivery.

Excipients may contain smaller amounts of additives as substances that increase the chemical isotonicity and stability of substances or buffers. The following are examples of buffers: phosphate buffer, bicarbonate buffer, Tris buffer; while timerosoi, m- or o-cresol, formalin and benzyl-alcohol are examples of preservatives. Standard formulations may be liquid or solid. Therefore, for a nonliquid formulation, excipients may contain dextrose, albumin of human serum, preservatives, etc. to which sterile saline solution may be added before delivery.

The present invention uses polymeric compounds: cyclodextrines, Liposomes, emulsions, all of which serve as carriers of the Tx2-6 toxin. These formulations may be administered via intramuscular injection, intravenous injection, subcutaneous injection, oral formulation, through inhalation or by means of devices including those directly implanted or injected into the corpus cavernosum.

No patent has been found in the state of the technique that would claim the use of the Tx2-6 toxin, its pharmaceutically acceptable salts as well as of pharmaceutical compounds of Tx2-6 based on cyclodextrines, mucoadhesive formulations or gels of topical application that can be used as a method of erectile function potentiation or for recovery of those suffering from erectile dysfunction, as for example, hypertensive individuals.

One of the features of the present invention is the use of Tx2-6 as an inducer of penile erection via NO release. This has been proved by in vitro and in vivo experiments in normotensive and hypertensive rats by using specific markers for NO (DAF-FM) and confocal microscopic analysis. Additionally, the present invention has also verified that the nonspecific NOS (L-NAME) inhibitor has completely blocked the potentiator effect of erection caused by the toxin, measured by means of the ICP/MAP relation, which has confirmed the central role played by NO in the toxin effect.

Another advantage of pharmaceutical compounds of the present invention with cyclodextrines, for example, is the prolonged action of the relaxation effect in corpus cavernosum tissues, the beginning of the effect as well as the increased facility of Tx2-6 delivery through mucoadhesive formulations and gels, based on carboxymethylcellulose as a nonlimiting example.

Interestingly, using Tx2-6 as an erection potentiating agent or even as an erectile function restorer for hypertensive individuals with affected function is due to the action mechanism of this molecule, which, according to experimental results, is distinguished from the major pharmaceutical used nowadays (sildenafil, Viagra®). Sildenafil citrate (scientific name IUPAC-citrate of 1-[4-etoxi-3-(6,7-dihidro-1-metil-7-oxo-3-propil-1H-pirazolo[4,3-d]pirimidin-5-il)fenilsulfonil)-4-metilpiperazina), traded as Viagra®, is a drug developed by Pfizer used for treating erectile dysfunction (male impotence) and lung arterial hypertension. Its main competitors in the drug market for the treatment of erectile dysfunction are tadalafila (Clalis®) and vardenafila (Levitra®, Vivanza+0).

Another important aspect is that the physiological process of erection partially involves the parasympathetic nervous system, which leads to release of nitric oxide (NO) in the penis corpus cavernosum. NO binds to the enzyme guanilate cyclase activating it, which results in increased levels of cyclic guanosine monophosphate (cGMP). cGMP induces relaxation of the corpus cavernosum smooth musculature (causing vasodilatation), which results in a greater blood inflow leading to erection.

Sildenafil (Viagra®) is a potent selective of type-5 phosphodiesterase specific to cGMP (PDE5), which is responsible for cGMP degradation in the penis corpus cavernosum. The molecular structure of sildenafil is similar to that of cGMP and it acts as a competitor with the latter in binding to PDE5 in the corpus cavernosum, resulting in this enzyme inhibition and therefore in higher availability of cGMP. Well-performed erections are achieved due to increased vasodilatation, generated by higher availability of cGMP. If there is no sexual stimulus and thus a deficient activation of the NO/cGMP system, sildenafil does not lead to erection. Other medicines acting through the same mechanism include tadalafila (Clalis®) and vardenafila (Levitra®). As for Tx2-6, it was confirmed that its mechanism of action is involved in the nitric oxide release, in this way interfering in a previous site, when compared to that where sildenafil acts. Therefore, as it acts on the voltage-dependent sodium channels of the nitrergic system, the toxin surely provokes depolarization, which would thus lead to the release of nitric oxide. Nitric oxide triggers the whole process as previously described, that is, it activates guanilate cyclase increasing the production of cGMP, which in turn leads to relaxation of the corpus cavernosum that determines erection. In brief: Tx2-6 releases nitric oxide, the primary factor activating erection. Viagra® constrains cGMP, which is formed by the primary action of nitric oxide and the last agent in the route, and allows that it be destroyed by phosphodiesterase 5. Therefore, we would propose another type of pharmaceutical, which could also be associated with other existing drugs, with the aim of correcting possible flaws in the system involved in the penile erection process.

The present invention can be better understood by means of the following, though nonlimiting, examples:

### EXAMPLE 1

### Animals and Drugs

All experiments accomplished have been approved by the Comitêde Ética em Experimentação Animal—CETEA (an ethical committee for experiments on animals) at the Universidade Federal de Minas Gerais (Federal Universty of Minas Gerais) (no.22/2006). Normotensive rats (12-14 weeks old, weighing 220-250 g) and hypertensive rats (CHITALEY, K., WEBB, R. C., DORRANCE, A. M., MILLS, T. M. Decreased penile erection in DOCA-salt and stroke prone-spontaneously hypertensive rats. International Journal of Impotence Research 13, 16-20, 2001) from the Biotério do Instituto de Ciências Biológicas (the Animal house of the Institute of Biological Sciences) of the Universidade Federal de Minas Gerais, MG, Brazil, were used in the experiments as model DOCA-sal. The normotensive animals had free access to standard food and water and were maintained in a 12 hr light/12 hr dark cycle, with light from 6 o'clock in the morning.

Wistar male rats (130-170 g) made hypertensive by unilateral nefrectomy and the implanting of an acetate “adhesive” of desoxicorticosterone (DOCA, 200 mg/Kg body weight) put onto the posterior part of the neck under anesthesia with tribromo-ethanol. DOCA rats received salted water (1% NaCl, 0.2% KCl) during 4 weeks. “Sham” control rats underwent unilateral nefrectomy and drank pure water. Systolic pressures were measured after 4 weeks of (DOCA/Sham) treatment. The effects of different doses of Tx2-6 on erectile function were measured in normotensive and hypertensive rats.

The participation of nitric oxide, resulting from the toxin effect on erection, was tested by using a nonselective inhibitor of the nitric oxide synthase enzyme, L-NAME (L-N^{G}-nitro-arginine methyl ester), intracavernosally injected (200 mg/Kg) in normotensive rats treated with Tx2-6 (subcutaneously injected; 48 μg/Kg).

### EXAMPLE 2

### Intracavernosal Pressure/Average Arterial Pressure (PIC/PAM) Measured In Vivo

Rats were anesthetized with urethane (140 mg/Kg, i.p) and put on a warmed platform. The left femoral artery was exposed and cannulated by using a 30 G needle connected to a tube (PE10) filled with heparinized saline, allowing a continuous monitoring of the median arterial pressure (MAP). The penile cavernosal nerve and corpus were exposed by means of median incision. The neighboring muscles were shifted to enable vision of the cavernosal nerve, which emerges from the ipsilateral ganglion and is located on the dorsal part of the prostate. A needle (30 G) connected to the tube (PE10) filled with heparinized saline was inserted into the corpus cavernosum base for measuring the intracavernosal pressure (ICP). The correct placing of the cannula was verified by observing a slight increase in intracavernosal pressure after insertion of cannula and tumescence response of the penile corpus following the heparinized saline inflow.

The arterial and cavernosal cannulas were connected to pressure transductors. The values were amplified by the MAP/ICP monitor and were expressed in mmHg. Pressure data were obtained and digitized at 12 Hz, visualized and recorded. After being isolated, the cavernosal nerve was placed in bipolar electrodes. Response curves to voltage (0.5, 3.0 V, 0.1 ms., 30 s each step) were made before and after (15 min) the subcutaneous injection of Tx2-6 (12 μg/Kg). Throughout all experiments, MAP/ICP records were taken after calibration of instruments.

### EXAMPLE 3

### Confocal Microscopy

The rats were anesthetized with an intraperitoneal injection of urethane (140 mg/kg) and sacrificed by abdominal artery bleeding. The corpora cavernosa of the normotensive rats were perfused with a 10 ml saline and the penises were then removed. Corpus cavernosum strips (approximately 3-12 mm long) were incubated with toxin Tx2-6 in different concentrations (0.1-0.01 μg/ml) and 2.5 μmolar DAF (DAF-FM Diacetate, 4-amino-5-methylamino-2′,7′-difluoroflurescein diacetate, Invitrogen Brasil Ltda) for 10 minutes. Then, the preparation was washed for three times with PBS (5 minutes/each washing) and frozen at −80° C. for 24 hr. Following this step, strips were moistened with OCT and cut (20 μm, 6 slices per animal) using a cryomicrotome at −20° C. The slices were fixed at ambient temperature on gelatinized sheets covered with glycerol (90%) and Tris-HCL buffer (10%) and kept frozen until confocal analysis (LSM-510) was carried out. Fluorescent images were obtained using a laser scanner (“argon-ion-laser”, oil-immersed objective, 63×) in a confocal microscope (excitation 488 nm). At least one image of each slice was captured and the nitric oxide, released by the cells, produced green fluorescence under confocal microscopy. The corpora cavernosa of normotensive rats that received 48 μg/Kg de Tx2-6 (i.v) were perfused with 10 μl of DAF (2.5 μmolar) after 10 minutes and immediately removed and prepared for analyses by confocal microscopy. The hypertensive rats (model DOCA-sal) and Sham also had their corpora cavernosa removed and analyzed by confocal microscopy after due preparation.

Results were expressed as a ±SEM average. The statistical analysis used the “two-way ANOVA” variance test followed by Bonferroni test. Differences of p<0.05 were considered statistically significant.

### EXAMPLE 4

### Effect of toxin Tx2-6 (Injected via Subcutaneous and Intravenous Routes) on Erection Function of Rats

Samples of toxin Tx2-6 (12 μg/kg) were injected via subcutaneous and intravenous routes in anesthetized rats, which were continuously monitored for median arterial pressure (MAP) and intracavernosal pressure (ICP), throughout the electrical stimulation of the major pelvic ganglion. The curves found were obtained by voltage variation (0.5-3.0 V, 12 Hz, 0.1 ms, 30 s each step). Such curves were obtained before and after 15 minutes of the toxin injection. The erectile response was significantly potentiated after subcutaneous injection of the toxin Tx2-6.

### EXAMPLE 5

### Toxin Tx2-6 Restores Erectile Function of Hypertensive Rats (DOCA-sal)

It is already known that hypertensive rats (model DOCA-sal) show severe erectile dysfunction as compared to the operated controls (Sham) (MILLS, T. M., CHITALEY, K., WINGARD, C. J., LEWIS, R. W., WEBB, R. C. Effect of Rho-kinase inhibition on vasoconstriction in the penile circulation. J Appl Physiol, 91, 1269-1273, 2001). The pretreatment (15 min) to which hypertensive rats had been submitted, with 12.0 μg/kg of Tx2-6 (s.c) injected into the ventral region, has induced an erectile function recovery of these rats. Their erectile function was continuously monitored during the experiment the preparation response to the ganglion electrical stimulus (ICP/AMP ratio).

### EXAMPLE 6

### Corpus Cavernosum Nitric Oxide Release of Rats in the Presence of Toxin Tx2-6

Nitric oxide is the most important neurotransmitter derived from nerves and endothelium involved in the erection process. For this reason, NO release in the penile tissue of rats was assessed using a specific fluorescent marker (DAF), which has the property of being luminous green in color in the presence of nitric oxide. This fluorescence was analyzed by means of confocal microscopy.

Different situations were observed and assessed: i) slices of penile tissue of normotensive rats were incubated in vitro with toxin Tx2-6 for 10 minutes in different concentrations; ii) in another series of experiments, this toxin was injected intravenously in normotensive rats and after 10 minutes OAF reagent was perfused in the corpora cavernosa, which were immediately removed and slices prepared; iii) the toxin was injected subcutaneously in both normotensive and hypertensive rats and 20 minutes later DAF reagent was injected in the corpora cavernosa and hence penile tissue was removed and slices were prepared.

### EXAMPLE 7

### Blockage of Enzyme Nitric Oxide Synthase (NOS) Hampers Tx2-6 Action of Erectile Function in Rats

Nitric oxide (NO) is generated from the amino acid L-arginine by means of the NOS enzyme action. This synthesis occurs in different parts of the organism, including the corpus cavernosum. In this experiment, an inhibitor nonspecific for NOS (L-NAME, 200 mg/Kg) was injected intracavemosally. It was verified that L-NAME blocked rat erectile function and that toxin Tx2-6 (12 μg/kg, s.c) was not able to revert it.

The present invention shows that the toxin Tx2-6 of Phoneutria nigriventer spider induces relaxation of rat corpus cavernosum by means of nitric oxide release. Relaxing substances, such as NO in smooth muscles, are synthesized in parassympatic nerve terminals and endothelial cells covering blood vessel walls and lacunar spaces in the corpus cavernosum (BURNETT, A. L., LOWENWTEIN, C. J., BREDT, D. S., CHANG, T. S., SNYDER, S. H. Nitric oxide: a physiologic mediator of penile erection. Science 257, 401-403, 1992).

In the present invention, a release of nitric oxide in the corpora cavernosa of rats was observed in the presence of the toxin Tx2-6, whose erection process caused by electrical stimulation (ICP/MAP ratio) was monitored in the presence or absence of the toxin. It was observed that toxin Tx2-6 caused significant potentiation of erectile function in anesthetized normotensive rats submitted to electrical stimulation of the pelvic ganglion. This result was clearly demonstrated in animals that had received the toxin via the subcutaneous route. Animals that had been injected intravenously also showed the toxin action, which was also confirmed in NO release visualized through confocal microscopy.

Erectile dysfunction in hypertensive rats (model DOCA-sal) is already known. Erectile function in these animals was totally recovered when toxin Tx2-6 was subcutaneously injected. The corpus cavernosum tissues of normotensive and hypertensive rats incubated in the presence of the toxin released nitric oxide, which was demonstrated by confocal microscopy analyses. The crucial role performed by nitric oxide, derived from “NANC” (nonadrenergic, noncholinergic) nerves and possibly from sinusoidal endothelium in the corpus cavernosum penile erection, is widely recognized (TODA N, AYAJIKI K, OKAMURA T. Nitric oxide and penile erectile function. Pharmacol Ther. 2005 May; 106(2):233-66).

The mechanisms involved in the contraction and relaxation of corpus cavernosum and penile vasculature have been intensively investigated in the last decades (ANDERSSON K. E., WAGNER G. Physiology of penile erection. Physiol Rev 75, 191-236, 1995). NO has been suggested as the main mediator of NANC nerves in the corpus cavernosum in vitro as well as in the cavernosal pressure in vivo in a variety of mammals, including rats (BURNETT, A. L., LOWENWTEIN, C. J., BREDT, D. S., CHANG, T. S., SNYDER, S. H. Nitric oxide: a physiologic mediator of penile erection. Science 257, 401-403, 1992), rabbits (TEIXEIRA, C. E., IFA, D. R., CORSO, G., SANTAGADA, V., CALIENDO, G., ANTUNES, E., de NUCCl, G. Sequence and structure-activity relationship of scorpion venom toxin with nitrergirc activity in rabbit corpus cavernosum. FASEB J, 17, 485-487, 2003), dogs (Hayashida H, Fujimoto H, Yoshida K, Tomoyoshi T, Okamura T, Toda N. Comparison of neurogenic contraction and relaxation in canine corpus cavernosum and penile artery and vein. Jon J. Pharmacol. 1996 November; 72(3):231-40), monkeys (OKAMURA T, AYAJIKI K, TODA N. Monkey corpus cavernosum relaxation mediated by NO and other relaxing factor derived from nerves. Am J. Physiol. 1998 April; 274(4 Pt 2):H1075-81) and humans (LEONE A M, WIKLUND N P, HOKFELT T, BRUNDIN L, MONCADA S. Release of nitric oxide by nerve stimulation in the human urogenital tract. Neuroreport. 1994 Feb. 24; 5(6):733-6).

Action potentials are known to open sodium channels sensitive to tetrodotoxin in nitrergic nerve terminals, which promote calcium inflow possibly through N-type Ca²⁺ channels within blood vessels. This seems to be the case in the corpus cavernosum as relaxing induced by electric stimulus of canine corpus cavernosum slices was sensitive to conotoxin, a specific blocker of N-type calcium channels (Leone et al, 1994; Okamura et al, 2001). Increased systolic calcium participates in the activation of nNOS in the presence of calmoduline (Bredt &Snyder, 1990). Experiments with rabbit corpus cavernosum suggest that NO neuronal release or synthesis of NO depends on calcium intracellular viability (Satio et al, 1993).

Isolation and purification of toxins, such as Tx2-6, provides important tools for the study of the action mechanism involved in penile erection, including nitrergic nerve stimulation, which results in NO release.

### EXAMPLE 8

### Preparation and Characterization of Inclusion Compounds of Tx2-6 in Cyclodextrines and the Action of Cyclodextrine-Included Tx2-6 in Rat Erectile Function

Preparing inclusion compounds between p-cyclodextrine and its derivatives and Tx2-6 and its pharmaceutically accepted salts in aqueous solutions in molar proportion ranging from 1:1 to 1:20 Tx2-6: β-cd and mechanical mixtures in the same nonlimiting molar proportions.

The preparation is made in molar proportions of β-cyclodextrine mentioned above and toxin Tx2-6 and its pharmaceutically accepted salts in aqueous solutions. The solution mixture is constantly agitated up to the complete β-cyclodextrine dissolution.

Afterwards, the mixture is frozen at the temperature of liquid nitrogen and is submitted to lyophilization for 24 hr. The solid matter thus obtained was characterized through physico-chemical techniques of analysis. The techniques providing important features of the host-guest interaction was circular dichroism, ITC, DSC, mass spectrometry (MALDI-TOF), fluorescence and absorption spectroscopy in the ultraviolet visible region.

The biological absorption and stability tests were carried out with toxin-cyclodextrine inclusion compound solutions. Experiments were carried out with cyclodextrine-included toxin and followed the same procedure as that used in non-included toxin (Example 4). Samples of cyclodextrine-included toxin Tx2-6 (12 μg/kg) were subcutaneously injected in anesthetized rats that were continuously monitored by median arterial (MAP) and intracavernosal (ICP) measures throughout electric stimulation of the major pelvic ganglion. The curves depicted were obtained by voltage variation (0.5-3.0 V, 12 Hz, 0.1 ms, 30 s at each step). These curves were obtained 15 minutes after the toxin injection. The erectile response was significantly potentiated after subcutaneous injection of toxin Tx2-6.

Therefore, cyclodextrine-included Tx2-6 allows an oral or systemic formulation with a longer erection effect. Such formulations may be encapsulated in mucoadhesive polymers or gels, which permit obtaining a topic formulation of Tx-2-6 and its use in potentiating erection in normotensive animals and in hypertensive animals for recovering their erectile activity.

### EXAMPLE 9

### Preparation and Characterization of Mucoadhesive Formulations and Gels Based on Hydroxylpromilmethyl Cellulose for Topic Application of Toxin Tx2-6 and Cyclodextrine-Included Compounds of Tx2-6

Preparing nonionic polymeric-based gel in polymers of the type hydroxyethyl cellulose and hydroxylpromilmethyl cellulose at 3% in cyclodextrine-included compounds of up to 1% Tx2-6.

For preparing these gels, methylparabene was solubilized in distilled water and left under agitation at a temperature of 70° C. A second solution containing an inclusion compound with cyclodextrine-included Tx2-6, glycol propylene and antioxydant (FDC—Vitamin C 1500 mg LOT 017G9, Nutro Laboratorie, inc.EUA) was prepared. These two solutions were mixed and left in repose up to 25° C. for the formation of gel.

| | |
|---|---|
| Distilled water | 100% |
| HEC or HPMC | 3% |
| Nipagin | 0.15% |
| Glycol Propylene | 15% |
| CD: Tx2-6 or Tx2-6 | 1% |
| Antioxydant 5% | q.s |

### EXAMPLE 10

### Preparation and Characterization of Mucoadhesive Formulations and Gels Based on Carpobol for Topic Application of Tx2-6 and Cyclodextrine-Included Compounds of Tx2-6

Preparation of Tx2-6 gel up to 1% in carbopol at 1% (ply) required the powder of this product to be dissolved in distilled water for 20 minutes with slow agitation. Solutions of Tx2-6 and its pharmaceutically acceptable salts were prepared up to 1% in glycol propylene (10%, 15% and 20%) and kept warmed at 70° C. Citric acid was slowly added to this solution, which was kept in agitation until reaching environment temperature. Both solutions were mixed under agitation and methylparabene was dissolved in absolute ethyl alcohol and added to the mixture. NaOH was added after complete homogeneization of ingredients as described below.

Preparation of carbopol gel at 1% of Tx2-6 and its inclusion compounds of up to 1%.

| | |
|---|---|
| Carbopol | 1% |
| Tx2-6 and/or inclusion compounds | 1% |
| Glycol propylene | 15% |
| Methylparabene | 0.15% |
| Ethylic alcohol | 50 μl |
| NaOH at 30% | 50 μl |
| Citric acid | 500 μl* |
| Distilled water | 100% |

*Final gel pH around 7.02.

### BRIEF DESCRIPTION OF FIGURES

FIG. 1. Penile erection induced by ganglionic stimulation in normotensive control rats after subcutaneous injection of Tx2-6. Ganglionic stimulus (0.5-3.0 V) induces an increased ICP/AMP ratio (intracavernosal pressure/median arterial pressure), which was significantly potentiated (FIG. 1A) by subcutaneous (n=6) or (FIG. 1B) intravenous (n=5) injection of Tx2-6 (12 μg/kg). * P<0.05 (A “Two-way” ANOVA test followed by Bonferroni test).

FIG. 2. The rat erectile function inhibited by L-NAME, a nonselective inhibitor of NOS (Nitric oxide synthase), is not recovered by Tx2-6. (FIG. 2A) ICP/AMP ratio (control) induced by submaximum ganglionic stimulation (1.5V). Ganglionic stimulation is significantly potentiated by Tx2-6 subcutaneous injection (FIG. 2B). Treatment with L-NAME (200 mg/Kg, intracavernosal) caused significant blockage an increase in ICP/AMP ratio induced by submaximum stimulation. This effect was not suppressed by subcutaneous injection of Tx2-6 (12 μg/kg). * P<0.05 (Two-way ANOVA followed by Bonferroni test).

FIG. 3. Increased release of NO (nitric oxide) in corpora cavernosa of normotensive control rats induced by Tx2-6 using the DAF-FM technique. FIG. 3A, incubation In vitro with Tx2-6 (0.01 μg/ml) induced NO release in cavernosal tissue slices. FIG. 3B, intensity of increased fluorescence in corpus cavernosum slices of rats incubated with Tx2-6. FIG. 3C, intravenous injection of Tx2-6 (48 μg/Kg) caused NO release in corpus cavernosum slices. FIG. 3D, increased fluorescence intensity observed in corpora cavernosa of normotensive control rats, which were intravenously injected with Tx2-6. Results are expressed as median±SEM; n=3. *P<0.05 (test t of Student).

FIG. 4. Penile erection induced by stimulation in hypertensive (DOCA-Sal) rats after Tx2-6 subcutaneous injection. FIG. 4A, ganglionic stimulation (0.5-3.0 V) induces an increased ICP/AMP ratio in rats Sham-operated, which was eased by subcutaneous injection of Tx2-6 (12 μg/kg, n=9). FIG. 4B, hypertensive DOCA-Sal rats exhibited a severe decreased ICP/AMP ratio under major pelvic ganglion (MPG) stimulation, which was totally recovered after a subcutaneous injection of Tx2-6 (12 μg/kg, n=6). * P<0.05 (Two-way ANOVA followed by Bonferroni test).

FIG. 5. Increased nitric oxide (NO) release induced by ganglionic stimulation of corpora cavernosa of Sham-operated rats and hypertensive DOCA-Sal rats treated with Tx2-6, using the DAF-FM technique. Injection of Tx2-6 (12.1 g/kg) potentiates an increase in NO release induced by ganglionic stimulation in cavernosal tissue of Sham-operated rats and hypertensive DOCA-Sal rats.

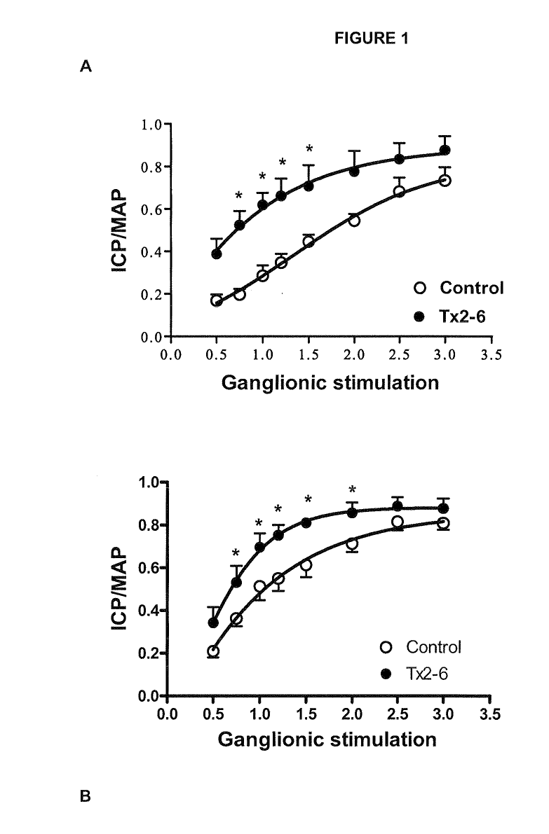

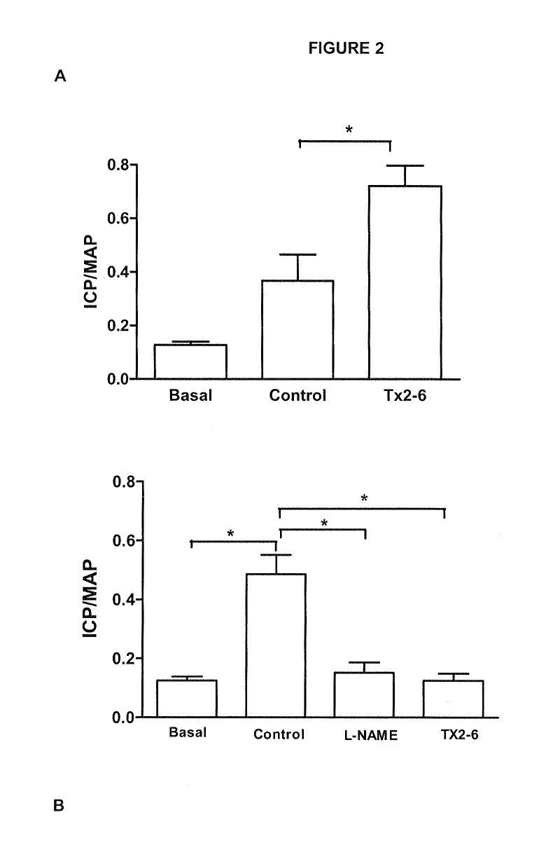

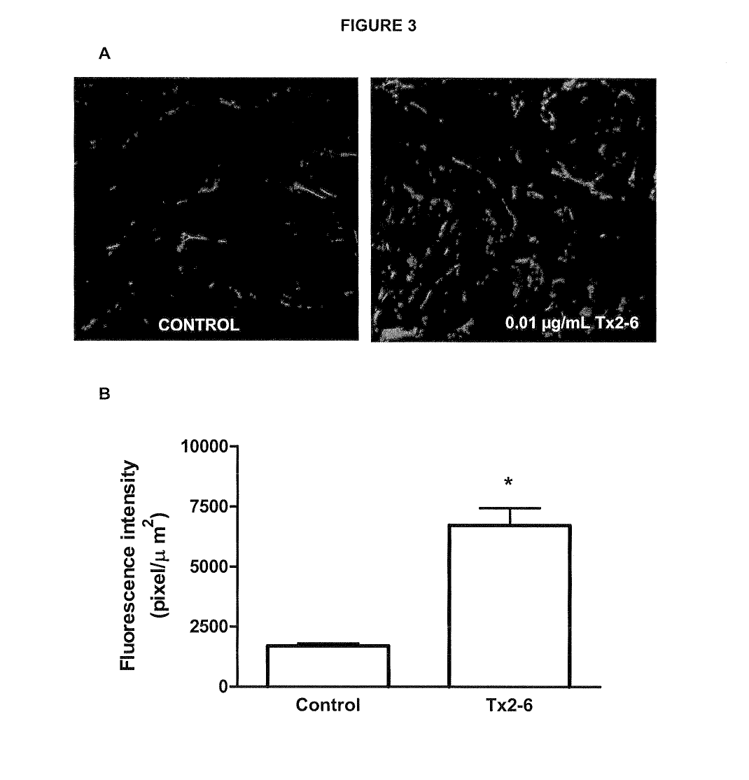

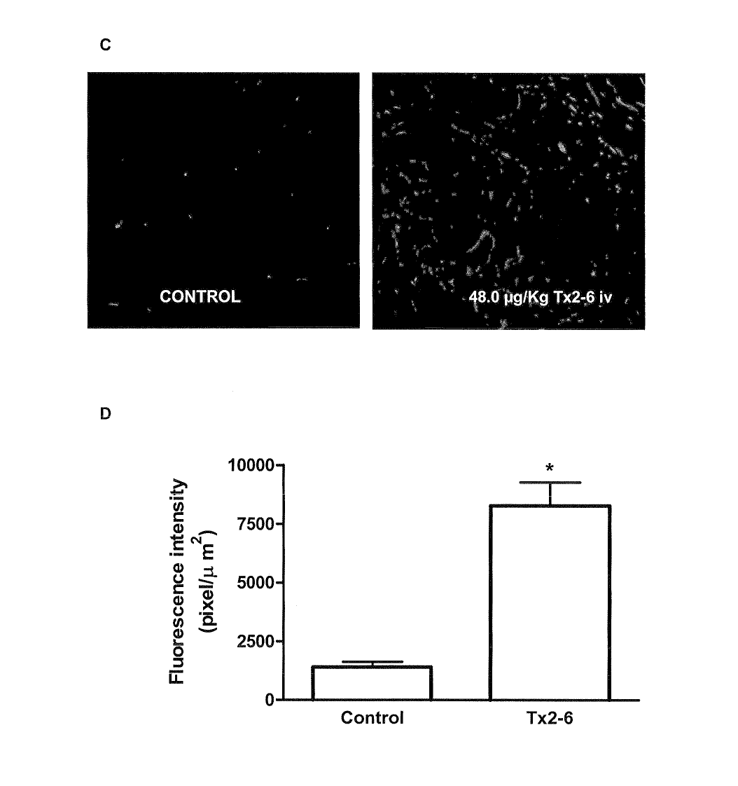

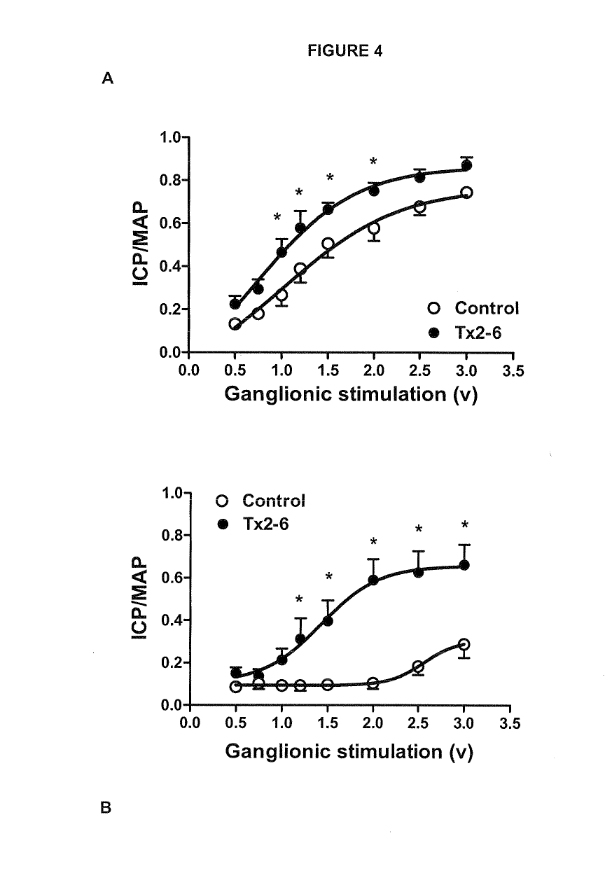

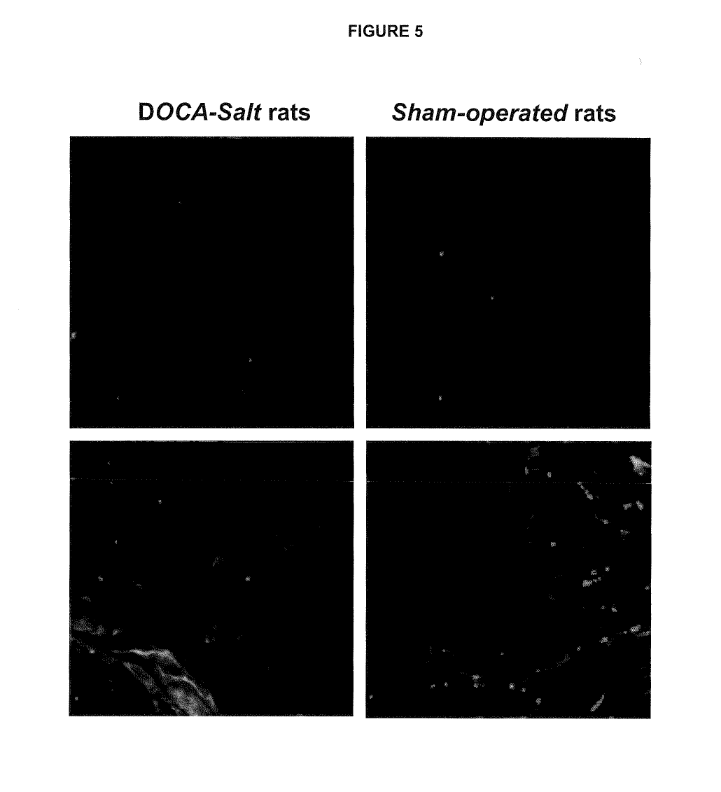

1. GENETICALLY MODIFIED SEQUENCE OF PHONEUTRIA NIGRIVENTER SPIDER TOXIN TX2-6, comprising DNA sequences SEQ ID no02 or SEQ ID no03.

2. PHARMACEUTICAL COMPOUND FOR ERECTILE FUNCTION POTENTIATION, according to claim 1, comprising the natural or synthetic toxin Tx2-6, its analogues or derivatives, in addition to pharmaceutically and physiologically acceptable carriers and excipients.

3. PHARMACEUTICAL COMPOUND FOR ERECTILE FUNCTION POTENTIATION, according to claim 2, comprising the amino acid sequence of toxin Tx2-6.

4. PHARMACEUTICAL COMPOUND FOR ERECTILE FUNCTION POTENTIATION, according to claim 3, wherein the natural toxin Tx2-6 is isolated from the Phoneutria nigriventer spider, sequence SEQ ID no01, or it is synthetically produced.

5. PHARMACEUTICAL COMPOUND FOR ERECTILE FUNCTION POTENTIATION, according to claim 4, comprising toxin Tx2-6 in an effective amount to potentiate the erectile function in mammals.

6. PHARMACEUTICAL COMPOUND FOR ERECTILE FUNCTION POTENTIATION, according to claim 5, which is delivered by oral, intramuscular, intravenous, subcutaneous, topic, transdermic routes or as devises to be implanted or injected even directly into the corpus cavernosum.

7. PHARMACEUTICAL COMPOUND FOR ERECTILE FUNCTION POTENTIATION, according to claim 6, comprising drug controlled release systems.

8. PHARMACEUTICAL COMPOUND FOR ERECTILE FUNCTION POTENTIATION, according to claim 7, comprising cyclodextrines, biodegradable polymers, mucoadhesive polymers, gels or liposomes.

9. METHOD OF ERECTILE FUNCTION POTENTIATION comprising the use of pharmaceutical compounds or formulations containing toxin Tx2-6.

10. METHOD OF ERECTILE FUNCTION POTENTIATION, according to claim 9, which stimulates nitric oxide release into the corpus cavernosum of mammals.

11. METHOD OF ERECTILE FUNCTION POTENTIATION, according to claim 10, which is applied to normotensive and hypertensive individuals.

12. USE OF PHARMACEUTICAL FORMULATIONS OF TOXIN TX2-6 for the manufacture of a medicament to potentiate the erectile function of mammals.


SEQUENCE LISTING (6 sequences)

SEQ ID NO: 1 - DNA, 323 bp, Phoneutria nigriventer
tcggcacgagatcagaatgaaagttgcaatcctcttcctctctattttggttgtgcttgctgcaagtgaatccattgaagaatcccgtgatgattttgctgtagaagaartggggagagccacatgcgctggccaagaccagccctgcaaagaaacttgcgactgctgtggagagagaggagaatgtgtttgcggaggaccttgcatttgcaggcaaggctacttttggatagcatggtataaacttgctaactgtaaaaaatgatatggatttatgtataccacgatataataaatatattgcatttgaaaaaaaaaaaaaa

SEQ ID NO: 2 - protein, 82 aa, Phoneutria nigriventer
MKVAILFLSILVVLAASESIEESRDDFAVEELGRATCAGQDQPCKETCDCCGERGECVCGGPCICRQGYFWIAWYKLANCKK

SEQ ID NO: 3 - DNA, 153 bp, Phoneutria nigriventer
gccatggccacatgcgctggccaagaccagccctgcaaagaaacttgcgactgctgtggagagagaggagaatgtgtttgcggaggaccttgcatttgcaggcaaggctacttttggatagcatggtataaacttgctaactgtaaaaaatga

SEQ ID NO: 4 - protein, 50 aa, Phoneutria nigriventer
AMATCAGQDQPCKETCDCCGERGECVCGGPCICRQGYFWIAWYKLANCKK

SEQ ID NO: 5 - DNA, 159 bp, Phoneutria nigriventer
ggttctgccatggccacatgcgctggccaagaccagccctgcaaagaaacttgcgactgctgtggagagagaggagaatgtgtttgcggaggaccttgcatttgcaggcaaggctacttttggatagcatggtataaacttgctaactgtaaaaaatga

SEQ ID NO: 6 - protein, 52 aa, Phoneutria nigriventer
GSAMATCAGQDQPCKETCDCCGERGECVCGGPCICRQGYFWIAWYKLANCKK